SEQUENCE LISTING (7 sequences)

SEQ ID NO: 1 - protein, 9 aa, Human immunodeficiency virus
RKKRRQRRR

SEQ ID NO: 2 - protein, 9 aa, Human immunodeficiency virus
RRRQRRKKR

SEQ ID NO: 3 - protein, 13 aa, Artificial Sequence
  Clostridum Botulinum
GDSCSVEAETAGK

SEQ ID NO: 4 - protein, 24 aa, Artificial Sequence
  Synthetic
RRRQRRKKRGGGDSCSVEAETAGK

SEQ ID NO: 5 - protein, 11 aa, Artificial Sequence
  Synthetic
RRRQRRKKRGG

SEQ ID NO: 6 - protein, 18 aa, Artificial Sequence
  Human Immunodeficiency Virus
RRRQRRKKRRRRQRRKKR

SEQ ID NO: 7 - protein, 11 aa, Artificial Sequence
  Synthetic
GGRRRQRRKKR

(19) United States
(12) Patent Application Publication
(10) Pub. No.: US 2008/0233152 A1
(43) Pub. Date: Sep. 25, 2008

(54) Compositions and Methods of Topical Application and Transdermal Delivery of Botulinum Toxins Stabilized with Polypeptide Fragments Derived from HIV-TAT

(75) Inventors: Jacob M. Waugh, Mountain View, CA (US); Jae Hoon Lee, Union City, CA (US)

Correspondence Address: KING & SPALDING, 1185 AVENUE OF THE AMERICAS, NEW YORK, NY, 10036-4003, US

(73) Assignee: REVANCE THERAPEUTICS, INC., Mountain View, CA (US)

(21) Appl. No.: 11/955,076

(22) Filed: Dec. 12, 2007

Related U.S. Application Data
(60) Provisional application No. 60/882,632, filed on Dec. 29, 2006.

(51) Int. Cl. A61K 39/08

(52) U.S. Cl. 424/239.1

(57) ABSTRACT

This invention relates to novel compositions of borulinum toxin that are stabilized using HIV-TAT fragments or derivatives of HIV-TAT fragments. The composition can be administered for various therapeutic, aesthetic and/or cosmetic purposes. The invention also provides method for stabilizing botulinum toxin using HIV-TAT fragments or derivatives or HIV-TAT fragments.

### CROSS-REFERENCES TO RELATED APPLICATIONS

This application claims priority to U.S. Provisional Application Ser. No. 60/882,632, filed Dec. 29, 2006, the contents of which are incorporated herein by reference in its entirety.

### FIELD OF THE INVENTION

This invention relates to novel compositions of botulinum toxin that can be applied

topically for various therapeutic, aesthetic and/or cosmetic purposes and that arc stabilized by polypeptide fragments derived from HIV-TAT.

### BACKGROUND OF THE INVENTION

Skin protects the body's organs from external environmental threats and acts as a

thermostat to maintain body temperature. It consists of several different layers, each with specialized functions. The major layers include the epidermis, the dermis and the hypodermis. The epidermis is a stratifying layer of epithelial cells that overlies the dermis, which consists of connective tissue. Both the epidermis and the dermis are further supported by the hypodermis, an internal layer of adipose tissue.

The epidermis, the topmost layer of skin, is only 0.1 to 1.5 millimeters thick (Inlander, Skin, New York, N.Y.: People's Medical Society, 1-7 (1998)). It consists of keratinocytes and is divided into several layers based on their state of differentiation. The epidermis can be further classified into the stratum comeum and the viable epidermis, which consists of me granular melphigian and basal cells. The stratum corncum is hygroscopic and requires at least 10% moisture by weight to maintain its flexibility and softness. The hygroscopicity is attributable in part to the water-holding capacity of keratin. When the horny layer loses its softness and flexibility it becomes rough and brittle, resulting in dry skin.

The dermis, which lies just beneath the epidermis, is 1.5 to 4 millimeters thick. It is the thickest of the three layers of the skin. In addition, the dermis is also home to most of the skin's structures, including sweat and oil glands (which secrete substances through openings in the skin called pores, or comedos), hair follicles, nerve endings, and blood and lymph vessels (Inlander, Skin, New York, N.Y.: People's Medical Society, 1-7 (1998)). However, the main components of the dermis are collagen and elastin.

The hypodermis is the deepest layer of the skin. It acts both as an insulator for body heat conservation and as a shock absorber for organ protection (Inlander, Skin, New York, N.Y.: People's Medical Society, 1-7 (1998)). In addition, the hypodermis also stores fat for energy reserves. The pH of skin is normally between 5 and 6. This acidity is due to the presence of amphoteric amino acids, lactic acid, and fatty acids from the secretions of the sebaceous glands. The term “acid mantle” refers to the presence of the water-soluble substances on most regions of the skin. The buffering capacity of the skin is due in part to these secretions stored in the skin's horny layer.

Wrinkles, one of the telltale signs of aging, can be caused by biochemical, histological, and physiologic changes that accumulate from environmental damage to the skin. (Benedetto, International Journal of Dermatology, 38:641-655 (1999)). In addition, there are other secondary factors mat can cause characteristic folds, furrows, and creases of facial wrinkles (Stegman et al., The Skin of the Aging Face Cosmetic Dermatological Surgery, 2^{nd} ed., St Louis, Mo.: Mosby Year Book: 5-15 (1990)). These secondary factors include the constant pull of gravity, frequent and constant positional pressure on the skin (e.g., during sleep), and repeated facial movements caused by the contraction of facial muscles (Stegman et al., The Skin of the Aging Face Cosmetic Dermatological Surgery, 2^{nd} ed., St. Louis, Mo.: Mosby Year Book: 5-15 (1990)).

Different techniques have been utilized in order to potentially mollify some of the signs of aging. These techniques range from facial moisturizers containing alpha hydroxy acids and retinol to surgical procedures and injections of neurotoxins. For example, in 1986, Jean and Alastair Carruthers, a husband and wife team consisting of an ocuplastic surgeon and a dermatologist, developed a method of using the type A form of botulinum toxin for treatment of movement-associated wrinkles in the glabella area (Schantz and Scott, In Lewis GE (Ed) Biomedical Aspects of Botulinum, New York: Academic Press, 143-150 (1981)). The Carruthers' use of the type A form of botulinum toxin for the treatment of wrinkles led to the seminal publication of this approach in 1992 (Schantz and Scott, In Lewis GE (Ed) Biomedical Aspects of Botulinum, New York: Academic Press, 143-150 (1981)). By 1994, the same team reported experiences with other movement-associated wrinkles on the face (Scott, Ophthalmol, 87:1044-1049 (1980)). This in turn led to the birth of the era of cosmetic treatment using the type A form of botulinum toxin.

Interestingly, the type A form of botulinum toxin is said to be the most lethal natural biological agent known to man. Spores of C. botulinum are found in soil and can grow in improperly sterilized and sealed food containers. Ingestion of the bacteria can cause botulism, which can be fatal. Botulinum toxin acts to produce paralysis of muscles by preventing synaptic transmission or release of acetylcholine across the neuromuscular junction, and is thought to act in other ways as well. Its action essentially blocks signals that normally would cause muscle spasms or contractions, resulting in paralysis. However, the muscle-paralyzing effects of botulinum toxin have been used for therapeutic effects. Controlled administration of botulinum toxin has been used to provide muscle paralysis to treat conditions, for example, neuromuscular disorders characterized by hyperactive skeletal muscles. Conditions that have been treated with botulinum toxin include hemifacial spasm, adult onset spasmodic torticollis, anal fissure, blepharospasm, cerebral palsy, cervical dystonia, migraine headaches, strabismus, temporomandibular joint disorder, and various types of muscle cramping and spasms. More recently the muscle-paralyzing effects of botulinum toxin have been taken advantage of in therapeutic and cosmetic facial applications such as treatment of wrinkles, frown lines, and other results of spasms or contractions of facial muscles.

In addition to the type A form of botulinum toxin, there are seven other serologically distinct forms of botulinum toxin that are also produced by the gram-positive bacteria Clostridium botulinum. Of these eight serologically distinct types of botulinum toxin, the seven that can cause paralysis have been designated botulinum toxin serotypes A, B, C (also known as C₁), D, E, F and G. Each of these is distinguished by neutralization with type-specific antibodies. The molecular weight of the botulinum toxin protein molecule, for all seven of these active botulinum toxin serotypes, is about 150 kD. The different serotypes of botulinum toxin vary in the animal species that they affect and in the severity and duration of the paralysis they evoke. For example, it has been determined that botulinum toxin type A is 500 times raoTe potent than botulinum toxin type B, as measured by the rate of paralysis produced in rats. Additionally, botulinum toxin type B has been determined to be non-toxic in primates at a dose of 480 U/kg, about 12 times the primate LD₅₀ for type A. Due to the molecule size and molecular structure of botulinum toxin, it cannot cross stratum corneum and the multiple layers of the underlying skin architecture.

As released by Clostridium botulinum bacteria, botulinum toxin is a component of a toxin complex containing the approximately 150 kD botulinum toxin protein molecule along with associated non-toxin proteins. These endogenous non-toxin proteins are believed to include a family of hemagglutinin proteins, as well as non-hcmagglutinin protein. The non-toxin proteins are believed to stabilize the botulinum toxin molecule in the toxin complex and protect it against denaturation, for example, by digestive acids when toxin complex is ingested. Thus, the non-toxin proteins of the toxin complex protect the activity of the botulinum toxin and enhance systemic penetration, particularly when the toxin complex is administered via the gastrointestinal tract. More specifically, it is believed that some of the non-toxin proteins specifically enhance penetration across the gastrointestinal epithelium while other non-toxin proteins stabilize the botulinum toxin molecule in blood. Additionally, the presence of non-toxin proteins in the toxin complexes typically causes the toxin complexes to have molecular weights that are greater than that of the bare botulinum toxin molecule, which is about 150 kD, as previously noted. For example, Clostridium botulinum bacteria can produce botulinum type A toxin complexes that have molecular weights of about 900 kD, 500 kD or 300 kD. Interestingly, botulinum toxin types B and C are apparently produced as only a 700 kD or a 500 kD complex. Botulinum toxin type D is produced as both 300 kD and 500 kD complexes. Botulinum toxin types E and F are produced as only approximately 300 kD complexes.

To provide additional stability to botulinum toxin, the toxin complexes arc often stabilized by combining them with exogenous stabilizers, (e.g., gelatin, polysaccharides, or most commonly additional albumin) during manufacturing. The stabilizers serve to bind and to stabilize toxin complexes in disparate environments, including those associated with manufacturing, transportation, storage, and administration.

Typically, the botulinum toxin is administered to patients by carefully controlled injections of compositions containing the botulinum toxin complex and albumin, but there are several problems associated with this approach. For example, because the injected toxin complexes contain non-toxin proteins and albumin, both of which stabilize the botulinum toxin and increase the molecular weight of the toxin complex, the toxin complexes have a long half-life in the body, are slow to diffuse through tissue, and may cause an undesirable antigenic response in the patient. Also, since the rion-toxin proteins and albumin stabilize the botulinum toxin in blood, the injections must be carefully placed so that they do not release a large amount of toxin into the bloodstream of the patient, which could lead to fatal systemic poisoning. Thus, injections typically must be performed precisely by highly trained medical professionals with a deep understanding of human anatomy.

In view of all of the problems discussed in the foregoing, it would be highly desirable to have a method of stabilizing botulinum toxin that does not use albumin. It would also be highly desirable if such a method were to reduce the antigenicity and blood stability of the botulinum toxin, while increasing the diffusion rate of botulinum toxin complexes within the body, thereby making it safer to use botulinum toxin for various therapeutic, aesthetic iand/or cosmetic purposes. It also would be desirable to have a method of administration that does not critically depend on precise injection of the bomlinum toxin by a medical professional in order to achieve safe administration of the toxin.

### SUMMARY OF THE INVENTION

One aspect of this invention is the recognition that certain polypeptide fragments of HIV-TAT, or polypeptide fragments derived from fragments of HIV-TAT, can be added to botulinum toxin complexes, and in particular reduced botulinum toxin complexes, to stabilize them. In a particularly preferred embodiment, the polypeptide fragment has as a sequence corresponding to amino acid residues 49-57 of HIV-TAT (RKKRRQRRR. SEQ ID NO. 1). In another preferred embodiment, the polypeptide fragment has a sequence corresponding to the reverse sequence of amino acid residues 49-57 of HIV-TAT (RRRQRRKKR, hereafter referred to as SEQ ID NO. 2.) Additionally, this invention also contemplates polypeptide analogs of the sequences of SEQ ID NOS 1 and 2 that are functionally equivalent, such as cases in which the conservative substitutions have been made. As used throughout this application, the reversed HIV-TAT polypeptide defined by SEQ ID NO. 2, as well as any polypeptide analog of SEQ ID NOS 1 or 2 in which conservative substitutions have been made, is encompassed by the term “HIV-TAT fragment derivative.”

Another aspect of this invention is the recognition that the endogenous non-toxin proteins in a botulinum toxin complex obtained from Clostridium botulinum bacteria (viz., the non-toxic hemagglutinin and non-hcmagghitinin proteins) undesirably increase the stability and toxicity of the toxin complex, while undesirably decreasing the ability of the toxin to diffuse through the skin epithelium. This invention further recognizes that these effects are exacerbated when an exogenous stabilizer, such as albumin, binds to botulinum toxin during conventional manufacturing processes. Thus, one aspect of this invention is to provide botulinum toxin complexes wherein the amounts of hemagglutinin, non-toxin non-hemagglutinin and/or exogenous albumin are selectively and independently reduced compared to conventional commercially available botulinum toxin (e.g., BOTOX® or MYOBLOC®). Such botulinum toxin complexes are hereafter referred to as “reduced botulinum toxin complexes”.

Accordingly, one object of this invention is to provide a composition comprising a botulinum toxin complex (or a reduced botulinum toxin complex) that is stabilized by polypeptides having a sequence corresponding to SEQ ID NO. 1 or 2. The composition optionally may contain added exogenous stabilizers, such as albumin.

As used herein, the term “stabilize” refers to the ability of the HIV-TAT fragments (e.g., SEQ ID NO. 1) or HIV-TAT fragment derivatives (e.g., SEQ ID NO. 2) to prevent the botulinum toxin from denaturing and to preserve the activity of the toxin, as measured by either a SNAPtide assay, or a Digital Abduction Scoring (DAS) assay. In preferred embodiments, the botulinum toxin compositions of this invention are sufficiently stabilized to retain substantially all of their biological activity during processing and patient administration steps, including, but not limited to, filling, lyophilizing, storing, and reconstituting for delivery.

The invention further relates to a method for producing a biologic effect by administering the stabilized botulinum complexes or stabilized reduced botulinum toxin complexes of the invention to a patient. In certain preferred embodiments, the stabilized botulinum complexes or stabilized reduced botulinum toxin complexes are topically applied in an effective amount, preferably to the skin, of a subject or patient in need of such treatment. The biologic effect may include, for example, muscle paralysis, reduction of hypersecretion or sweating, treatment of neurologic pain or migraine headache, reduction of muscle spasms, prevention or reduction of acne, reduction or enhancement of an immune response, reduction of wrinkles, or prevention or treatment of various other disorders. In other embodiments, the stabilized botulinum toxin complexes or stabilized reduced botulinum toxin complexes are administered by parenteral injection, such as, for example, subcutaneous injection.

This invention also provides kits for preparing formulations containing a botulinum toxin complex (or a reduced botulinum toxin complex) and polypeptides having sequences according to SEQ ID NOS, 1 or 2, or a premix that may in turn be used to produce such a formulation. Also provided are kits that contain means for sequentially administering a botulinum toxin complex (or a reduced botulinum toxin complex) and adhesion molecules to a subject.

### DETAILED DESCRIPTION OF THE INVENTION

This invention relates to novel compositions comprising botulinum toxin complexes or reduced botulinum toxin complexes, as described herein, that are stabilized by the addition of polypeptides that are HIV-TAT fragments or HIV-TAT fragment derivatives. In preferred embodiments, the stabilizing polypeptides have a sequence according to SEQ ID NOS 1 or 2, or may be related to (hose sequences through conservative substitutions. In certain embodiments, the stabilized botulinum toxin compositions according to the invention enable the transport or delivery of a botulinum toxin through the skin epithelium (also referred to as “transdermal delivery”) with improved penetration, reduced antigenicity and blood stability. The compositions of the invention may be used as topical applications for providing a botulinum toxin to a subject, for various therapeutic, aesthetic and/or cosmetic purposes, as described herein. The compositions of the invention also have an improved safety profile over other compositions and methods of delivery of botulinum toxin.

The term “botulinum toxin” as used herein refers to any of the known types of botulinum toxin (i.e., the approximately 150 kD botulinum toxin protein molecule), whether produced by the bacterium or by recombinant techniques, as well as any such types that may be subsequently discovered including newly discovered serotypes, and engineered variants or fusion proteins. As mentioned above, currently seven immunologically distinct botulinum neurotoxins have been characterized, namely botulinum neurotoxin serotypes A, B, C, D, E, F and G, each of which is distinguished by neutralization with type-specific antibodies. The botulinum toxin serotypes are commercially available, for example, from Sigma-Aldrich (St. Louis, Mo.) and from Metabiologics, Inc. (Madison, Wis.), as well as from other sources. The different serotypes of botulinum toxin vary in the animal species that they affect and in the severity and duration of the paralysis they evoke. At least two types of botulinum toxin, types A and B, are available commercially in formulations for treatment of certain conditions. Type A, for example, is contained in preparations of Allergan having the trademark BOTOX® and of Ipsen having the trademark DYSPORT®, and type B is contained in preparations of Elan having the trademark MYOBLOC®.

The term “botulinum toxin” used in the compositions of this invention can alternatively refeT to a botulinum toxin derivative, that is, a compound that has botulinum toxin activity but contains one or more chemical or functional alterations on any part or on any chain relative to naturally occurring or recombinant native botulinum toxins. For instance, the botulinum toxin may be a modified neurotoxin that is a neurotoxin that has at least one of its amino acids deleted, modified or replaced, as compared to a native, or the modified neurotoxin can be a recombinantly produced neurotoxin or a derivative or fragment thereof. In one particularly preferred embodiment of the invention, the botulinum toxin derivative is a polypeptide having the sequence GDSCSVEAETAGK (SEQ ID NO. 3). This sequence corresponds to the portion of the type A botulinum toxin molecule that is responsible for the toxin's biological activity in humans. The botulinum toxin may also be one that has been modified in a way that, for instance, enhances its properties or decreases undesirable side effects, but that still retains the desired botulinum toxin activity. The botulinum toxin may be from any of the botulinum toxin complexes produced by the bacterium, as described above. Alternatively the botulinum toxin used in this invention may be a toxin prepared using recombinant or synthetic chemical techniques, e.g. a recombinant peptide, a fusion protein, or a hybrid neurotoxin, for example prepared from subunits or domains of different botulinum toxin serotypes (see U.S. Pat. No. 6,444,209, for instance). The botulinum toxin may also be a portion of the overall molecule that has been shown to possess the necessary botulinum toxin activity, and in such case may be used per se or as part of a combination or conjugate molecule, for instance a fusion protein. Alternatively, the botulinum toxin may be in the form of a botulinum toxin precursor, which may itself be non-toxic, for instance a nontoxic zinc protease that becomes toxic on proteolytic cleavage.

The term “botulinum toxin complex” or “toxin complex” as used herein refers to a botulinum toxin (e.g, the approximately 150 kD botulinum toxin protein molecule belonging to any one of botulinum toxin serotypes A-G, or the botulinum toxin fragment of SEQ ID NO. 3), along with associated endogenous non-toxin proteins (i.e., hemagglutinin protein and non-toxin non-hemagglutinin protein produced by Clostridium botulinum bacteria). Note, however, that the botulinum toxin complex need not be derived from Clostridium botulinum bacteria as one unitary toxin complex. For example, botulinum toxin or modified botulinum toxin may be recombinantly prepared first and then subsequently combined with the non-toxin proteins. Recombinant botulinum toxin can be also be purchased (e.g., from List Biological Laboratories, Campbell, Calif.) and then combined with non-toxin proteins.

This invention also contemplates “reduced botulinum toxin complexes”, in which the botulinum toxin complexes (including those that contain botulinum toxin derivatives, such as the polypeptide sequence in SEQ ID NO. 3) have reduced amounts of non-toxin protein compared to the amounts naturally found in botulinum toxin complexes produced by Clostridium botulinum bacteria. In one embodiment, reduced botulinum toxin complexes are prepared using any conventional protein separation method to extract a fraction of the hemagglutinin protein or non-toxin non-hemagglutinin protein from botulinum toxin complexes derived from Clostridium botulinum bacteria. For example, reduced botulinum toxin complexes may be produced by dissociating botulinum toxin complexes through exposure to red blood cells at a pH of 7.3 (e.g., see EP 1514556 A1, hereby incorporated by reference). HPLC, dialysis, columns, cenrrifugation, and other methods for extracting proteins from proteins can be used. Alternatively, when the reduced botulinum toxin complexes are to be produced by combining synthetically produced botulinum toxin with non-toxin proteins, one may simply add less hemagglutinin or non-toxin non-hemagglutinin protein to the mixture than what would be present for naturally occurring botulinum toxin complexes. Any of the non-toxin proteins (e.g., hemagglutinin protein or non-toxin non-hemagglutinin protein or both) in the reduced botulinum toxin complexes according to the invention may be reduced independently by any amount In certain exemplary embodiments, one or more non-toxin proteins are reduced by at least about 0.5%, 1%, 3%, 5%, 10%, 20%, 30%, 40%, 50%, 60% 70%, 80% or 90% compared to the amounts normally found in botulinum toxin complexes. MYOBLOC has 5000 U of Botulinum toxin type B per ml with 0.05% human serum albumin, 0.01 M sodium succinate, and 0.1 M sodium chloride. DYSPORT has 500 U of botulinum toxin type A-hemagglutinin complex with 125 mcg albumin and 2.4 mg lactose. In one particularly interesting embodiment, substantially all of the non-toxin protein (e.g., >95% of the hemagglutinin protein and non-toxin non-hemagglutinin protein) that would normally be found in botulinum toxin complexes derived from Clostridium botulinum bacteria is removed from the botulinum toxin complex. Furthermore, although the amount of endogenous non-toxin proteins may be reduced by the same amount in some cases, this invention also contemplates reducing each of the endogenous non-toxin proteins by different amounts, as well as reducing at least one of the endogenous non-toxin proteins, but not the others.

In addition to (or instead of) reducing the amount of endogenous non-toxin protein to destabilize the botulinum toxin complex, this invention also contemplates reducing the amount of exogenous stabilizers that are normally added during manufacturing. An example of such an exogenous stabilizer is albumin, which is normally added during manufacturing to botulinum toxin complexes in amount equal to 1000 times the amount of albumin found in the endogenous non-toxin, non-hemagglutinin component of a naturally occurring botulinum toxin complex. According to this invention, the amount of added exogenous albumin can be any amount less than the conventional thousand-fold excess of exogenous albumin. In certain exemplary embodiments of the invention, only about 500×, 400×, 300×, 200×, 100×, 50×, 10×, 5×, 1×, 0.5×, 0.1×, or 0.01× the amount of the albumin in naturally occurring botulinum toxin complexes is added. In one embodiment, no exogenous albumin is added as a stabilizer to the compositions of the invention. In other embodiments, exogenous stabilizers in addition to (or instead of) albumin arc added to the therapeutic topical compositions of the invention. For example, other stabilizers contemplated by the invention include lactose, gelatin and polysaccharides.

While the stabilized botulinum toxin complexes or stabilized reduced botulinum toxin complexes can be obtained or derived from any of the botulinum toxin serotypes (i.e., types A-G), in preferred embodiments of this invention, they are obtained or derived from the type A serotype of botulinum toxin.

In preferred embodiments, the botulinum toxin compositions of the invention are stabilized by the addition of non-native polypeptides that are either a fragment of HIV-TAT (e.g., SEQ ID NO. 1) or derived from a fragment of HIV-TAT (e.g., SEQ ID. NO 2, which is the reverse sequence of the polypeptide of SEQ ID NO. 1). The HIV-TAT fragment or derivative thereof may be combined with the botulinum toxin molecule cither covalently or non-covalently to stabilize the botulinum toxin complex or reduced botulinum toxin complex. In one preferred embodiment, the HIV-TAT fragment or derivative thereof is physically combined with botulinum toxin complexes or reduced botulinum complexes to stabilize them non-covalently. The relative amount of HIV-TAT fragment or derivative thereof will depend on the degree of stability desired. For example, when the stabilizing HIV-TAT fragment or derivative thereof corresponds to the polypeptides of SEQ ID NOs. 1 or 2, a useful concentration range for the stabilizing peptide about 0.1 ng to about 1.0 mg per unit of the botulinum toxin complex or reduced botulinum toxin complex. More preferably, the stabilizing peptides of SEQ ID NOs. 1 or 2 can be in the range of about 0.1 mg to 0.5 mg per unit of botulinum toxin.

Alternatively, the stabilizing HIV-TAT fragment or derivative thereof can be covalently linked to the botulinum toxin molecule in a botulinum toxin complex or reduced botulinum toxin complex using linking chemistry known in the art. By way of example, coupling of the two constituents can be accomplished via a coupling or conjugating agent. There are several intermolecular cross-linking reagents that can be utilized (see, for example, Means, G. E. and Feeney, R. E., Chemical Modification of Proteins, Holden-Day, 1974, pp. 39-43). Among these reagents are, for example, J-succinimidyl 3-(2-pyridyldithio) propionate (SPDP) or N,N′-(1,3-phenylene) bismaleimide (both of which are highly specific for sulfhydryl groups and form irreversible linkages); N,N′-ethylene-bis-(iodoacetamide) or other such reagent having 6 to 11 carbon methylene bridges (which relatively specific for sulfhydryl groups); and 1,5-difluoro-2,4-dinitrobenzene (which forms irreversible linkages with amino and tyrosine groups). Other cross-linking reagents useful for this purpose include: p,p′-difluoro-m,m′-dinitrodiphenylsulfone (which forms irreversible cross-linkages with amino and phenolic groups); dimethyl adipimidatc (which is specific for amino groups); phenol-1,4-disulfonylchloride (which reacts principally with amino groups); hexamethylcnediisocyanate or diisothiocyanate, or azophenyl-p-diisocyanate (which reacts principally with amino groups); glutaraldehyde (which reacts with several different side chains) and disdiazobenzidine (which reacts primarily with tyrosine and histidine).

Cross-linking reagents may be homobifunctional, i.e., having two functional groups that undergo the same reaction. A preferred homobifunctional cross-linking reagent is bismaleimidohexane (“BMH”). BMH contains two maleimide functional groups, which react specifically with sulfhydryl-containing compounds under mild conditions (pH 6.5-7.7). The two maleimide groups are connected by a hydrocarbon chain. Therefore, BMH is useful for irreversible cross-linking of polypeptides that contain cysteine residues.

Cross-linking reagents may also be heterobifunctional. Heterobifunctional cross-linking agents have two different functional groups, for example an amine-reactive group and a thiol-reactive group, that will cross-link two proteins having free amines and thiols, respectively. Examples of heterobifunctional cross-linking agents are succinimidyl 4-(N-maleimidomethyl)cyclohexane-1-carboxylatc (“SMCC”), m-malcimidobenzoyl-N-hydroxysuccinimide ester (“MBS”), and succinimidc 4-(p-maleimidophenyl)buryrate (“SMPB”), an extended chain analog of MBS. The succinimidyl group of these cross-linkers reacts with a primary amine, and the thiol-reactive maleimide forms a covalent bond with the thiol of a cysteine residue.

Cross-linking reagents often have low solubility in water. A hydrophilic moiety, such as a sulfonate group, may be added to the cross-linking reagent to improve its water solubility. Sulfo-MBS and sulfo-SMCC are examples of cross-linking reagents modified for water solubility.

Many cross-linking reagents yield a conjugate that is essentially non-cleavable under cellular conditions. However, some cross-linking reagents contain a covalent bond, such as a disulfide, that is cleavable under cellular conditions. For example, dithiobis(succinimidylpropionate) (“DSP”), Trau's reagent and N-succinimidyl 3-(2-pyridyldithio) propionate (“SPDP”) are well-known cleavable cross-linkers. The use of a cleavable cross-linking reagent permits the stabilizing HIV-TAT fragment or derivative thereof to separate from the botulinum toxin molecule/after delivery into the target area. Direct disulfide linkage may also be useful.

Some new cross-linking reagents such as n-γ-maleimidoburyryloxy-succmimide ester (“GMBS”) and sulfo-GMBS, have reduced immunogenicity. In some embodiments of the present invention, such reduced immunogenicity may be advantageous.

Numerous cross-linking reagents, including the ones discussed above, are commercially available. Detailed instructions for their use are readily available from the commercial suppliers. A general reference on protein cross-linking and conjugate preparation is: S. S. Wong, Chemistry of Protein Conjugation and Cross-Linking, CRC Press (1991).

Chemical cross-linking may include the use of spacer arms. Spacer arms provide intramolecular flexibility or adjust intramolecular distances between conjugated moieties and thereby may help preserve biological activity. A spacer arm may be in the form of a polypeptide moiety comprising spacer amino acids. Alternatively, a spacer arm may be part of the cross-linking reagent, such as in “long-chain SPDP” (Pierce Chem. Co., Rockford, Ill., cat. No. 21651 H).

In addition to chemical linking to produce stabilized botulinum toxin complexes or reduced botulinum toxin complexes, this invention also contemplates using genetic fusion techniques to produce these stabilized toxin complexes. For example, using well-known genetic engineering techniques, the nucleic acid sequences that code for a fused botulinum toxin/HIV-TAT fragment or a botulinu toxin/HIV-TAT fragment derivative can be implanted into cells, to cause the cells to express the stabilized toxin complexes.

In particularly preferred embodiments of this invention, the HIV-TAT fragment or HIV-TAT fragment derivative is covalently attached to the end of the botulinum toxin molecule or derivative thereof to form a linear molecule. In such embodiments, it is often advantageous to use glycine spacers between the botulinum toxin (or derivative thereof) and the HIV-TAT fragment or HIV-TAT fragment derivative. For example, when the botulinum toxin derivative is the polypeptide according to SEQ ID NO. 3, the stabilized botulinum toxin may have the form RRRQRRKKR-GG-GDSCSVEAETAGK (SEQ ID NO. 4). When it is desired to add more than one stabilizing polypeptide to a toxin molecule, the stabilized botulinum toxin may have the form RRRQRRKKR-GG-toxin amino acids-GG-RRRQRRKKR. Note however, that this invention also contemplates the use of repeating units of HIV-TAT fragments or derivatives thereof (e.g., RRRQRRKKR RRRQRRKKR) for stabilization, cither by covalent or non-covalent attachment.

The number of stabilizing polypeptide chains (whether they are HIV-TAT fragments or derivatives thereof) that are needed to stabilize a botulinum toxin molecule will depend on factors such as the particular serotype in question, and the size and chemical composition of the botulinum toxin or botulinum toxin fragment or derivative under consideration. For example, when a botulinum toxin derivative is being used and it is a relatively small polypeptide (e.g., the polypeptide according to SEQ ID NO. 3), fewer stabilizing polypeptide chains need to be covalently attached, and one covalently attached stabilizing polypeptide chain (e.g., the polypeptide of SEQ ID NOs 1 or 2, or derivatives thereof) may suffice for certain applications.

Compositions of this invention are preferably in the form of products to be applied to the skin or epithelium of subjects or patients, i.e. humans or other mammals in need of the particular treatment. The term “in need” is meant to include both pharmaceutical or health-related needs, for example, treating conditions involving undesirable facial muscle spasms, as well as cosmetic and subjective needs, for example, altering or improving the appearance of facial tissue. Generally, the compositions of this invention can be applied by any means known in the art, non-limiting examples of which include parenteral injection (eg., subcutaneous injection), topical administration on a skin, or via a patch that can be sub-dermally or supra-dermally located.

The HIV-TAT fragment of SEQ ID NO. 1 has been previously recognized as promoting intracellular delivery of various “cargo molecules” (see, e.g., U.S. Pat. No. 5,804,604). Thus, when botulinum toxin complexes or reduced botulinum toxin complexes have been stabilized with the HIV-TAT fragment of SEQ ID NO. 1 contacts the tissues of a patient (e.g., during topical administration), enhanced cellular penetration of botulinum toxin occurs. In addition, the HIV-TAT derived polypeptide having the sequence of SEQ ID NO. 2 also promotes intracellular penetration, as well as transmembrane penetration. Accordingly, enhanced intracellular and/or transmembrane transport of botulinum toxin occurs when botulinum toxin complexes or reduced botulinum toxin complexes that have been stabilized with the polypeptide of SEQ ID NO. 2 contacts the tissues of a patient

In general, the compositions of the invention are prepared by mixing the stabilized botulinum toxin complexes or stabilized reduced botulinum toxin complexes with one or more additional pharmaceutically acceptable carriers or excipients. In their simplest form they may contain a simple aqueous pharmaceutically acceptable carrier or diluent, such as buffered saline. Such embodiments are particularly preferred when the compositions of the invention are to be administered by injection. However, when the compositions of the invention are to be applied topically, they may contain other ingredients typical in topical pharmaceutical or cosmeceutical compositions, that is, a dermatologically or pharmaceutically acceptable carrier, vehicle or medium, i.e. a carrier, vehicle or medium that is compatible with the tissues to which they will be applied. The term “dermatologically or pharmaceutically acceptable,” as used herein, means that the compositions or components thereof so described are suitable for use in contact with these tissues or for use in patients in general without undue toxicity, incompatibility, instability, allergic response, and the like. As appropriate, compositions of the invention may comprise any ingredient conventionally used in the fields under consideration, and particularly in cosmetics and dermatology.

In terms of their form, compositions of this invention may include solutions, emulsions (including microemulsions), suspensions, creams, lotions, gels, powders, or other typical solid or liquid compositions used for application to skin and other tissues where the compositions may be used. Such compositions may contain, in addition to the botulinum toxin and HIV-TAT fragments or derivatives thereof, other ingredients typically used in such products, such as antimicrobials, moisturizers and hydration agents, penetration agents, preservatives, emulsifiers, natural or synthetic oils, solvents, surfactants, detergents, gelling agents, emollients, antioxidants, fragrances, fillers, thickeners, waxes, odor absorbers, dyestuffs, coloring agents, powders, viscosity-controlling agents and water, and optionally including anesthetics, anti-itch actives, botanical extracts, conditioning agents, darkening or lightening agents, glitter, humectants, mica, minerals, polyphenols, silicones or derivatives thereof, sunblocks, vitamins, and phytomcdicinals.

Compositions according to this invention may be in the form of controlled-release or sustained-release compositions, wherein the stabilized botulinum toxin complexes or stabilized reduced botulinum toxin complexes are encapsulated or otherwise contained within a material such that they are released onto the skin in a controlled manner over time. The composition comprising the botulinum toxin and HIV-TAT fragments or derivatives thereof molecules may be contained within matrixes, liposomes, vesicles, microcapsules, microspheres and the like, or within a solid particulate material, all of which is selected and/or constructed to provide release of the stabilized botulinum toxin over time.

Botulinum toxin can be delivered to muscles underlying the skin, or to glandular structures within the skin, in an effective amount to produce paralysis, produce relaxation, alleviate contractions, prevent or alleviate spasms, reduce glandular output, or other desired effects. Local delivery of the botulinum toxin in this manner could afford dosage reductions, reduce toxicity and allow more precise dosage optimization for desired effects relative to injectable or implantable materials.

The compositions of the invention are applied so as to administer an effective amount of the botulinum toxin. The term “effective amount” as used herein means an amount of a botulinum toxin as defined above that is sufficient to produce the desired muscular paralysis or other biological or aesthetic effect, but that implicitly is a safe amount, i.e. one that is low enough to avoid serious side effects. Desired effects include the relaxation of certain muscles with the aim of, for instance, decreasing the appearance of fine lines and/or wrinkles, especially in the face, or adjusting facial appearance in other ways such as widening the eyes, lifting the corners of the mouth, or smoothing lines that fan out from the upper lip, or the general relief of muscular tension. The last-mentioned effect, general relief of muscular tension, can be effected in the face or elsewhere. The compositions of the invention may contain an appropriate effective amount of the botulinum toxin for application as a single-dose treatment, or may be more concentrated, either for dilution at the place of administration or for use in multiple applications. The stabilized botulinum toxin complexes or stabilized reduced botulinum toxin complexes can be administered transdermally to a subject for treating conditions such as undesirable facial muscle or other muscular spasms, hyperhidrosis, acne, or conditions elsewhere in the body in which relief of muscular ache or spasms is desired. The botulinum toxin is administered topically for transdermal delivery to muscles or to other skin-associated structures. The administration may be made, for example, to the legs, shoulders, back (including lower back), axilla, palms, feet, neck, groin, dorsa of the hands or feet, elbows, upper arms, knees, upper legs, buttocks, torso, pelvis, or any other part of the body where administration of the botulinum toxin is desired.

Administration of botulinum toxin may also be carried out to treat other conditions, including but not limited to treating neurologic pain, prevention or reduction of migraine headache or other headache pain, prevention or reduction of acne, prevention or reduction of dystonia or dystonic contractions (whether subjective or clinical), prevention or reduction of symptoms associated with subjective or clinical hypcrhidrosis, reducing hypersecretion or sweating, reducing or enhancing immune response, or treatment of other conditions for which administration of botulinum toxin by injection has been suggested or performed.

Most preferably, the compositions are administered by or under the direction of a physician or other health care professional. They may be administered in a single treatment ot in a series of periodic treatments over time. For transdermal delivery of botulinum toxin for the purposes mentioned above, a composition as described above is applied topically to the skin at a location or locations where the effect is desired. Because of its nature, most preferably the amount of botulinum toxin applied should be applied with care, at an application rate and frequency of application that will produce the desired result without producing any adverse or undesired results. Accordingly, for instance, topical compositions of the invention should be applied at a rate of from about 1U to about 20,000U, preferably from about 1U to about 2.000U botulinum toxin per cm² of skin surface. Higher dosages within these ranges could preferably be employed in conjunction with controlled release materials, for instance, or allowed a shorter dwell time on the skin prior to removal.

This invention also includes transdermal delivery devices for transmitting botulinum toxin-containing compositions described herein across skin. Such devices may be as simple in construction as a skin patch, or may be a more complicated device that includes means for dispensing and monitoring the dispensing of the composition, and optionally means for monitoring the condition of the subject in one or more aspects, including monitoring the reaction of the subject to the substances being dispensed.

The compositions of this invention are suitable for use in physiologic environments with pH ranging from about 4.5 to about 6.3, and may thus have such a pH. The compositions according to this invention may be stored either at room temperature or under refrigerated conditions.

It is understood that the following examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

We claim:

1. A method for stabilizing botulinum toxin, said method comprising
providing a botulinum toxin complex or a reduced botulinum toxin complex;
providing an polypeptide that is an HIV-TAT fragment or an HIV-TAT fragment derivative, and
combining said botulinum toxin complex or reduced botulinum toxin complex with said polypeptide.

2. The method according to claim 1, wherein said botulinum toxin complex or reduced botulinum toxin complex is covalently attached to said polypeptide.

3. The method of claim 1, wherein said botulinum toxin complex or reduced botulinum toxin complex is non-covalently stabilized by said polypeptide.

4. The method of claim 1, wherein said HIV-TAT fragment has a sequence according to SEQ ID NO. 1.

5. The method of claim 1, wherein said HIV-TAT fragment has a sequence according to SEQ ID NO. 2.

6. The method of claim 1, wherein said botulinum toxin complex or reduced botulinum toxin complex comprises a polypeptide having a sequence according to SEQ ID NO. 3.

7. The method of claim 1, wherein the reduced botulinum toxin complex contains a reduced amount of hemagglutinin protein or non-toxin, non-hemagglulinin protein or both compared to an amount naturally occurring in botulinum toxin complexes directly extracted from Clostridium botulinum.

8. The method according to claim 1, wherein the botulinum toxin complex or reduced botulinum toxin complex contains albumin as an exogenous stabilizer.

9. The method of claim 6, wherein the albumin is present in an amount equal to about 500, 400, 300, 200, 100, 50, 10, 5, 1, 0.5, 0.1, or 0.01 times the amount of the albumin in naturally occurring botulinum toxin complexes

10. The method of claim 1, wherein the botulinum toxin complex or reduced botulinum toxin complex contains a botulinum toxin selected from the group consisting of a botulinum toxin derivative, a recombinant botulinum toxin, a modified botulinum toxin, botulinum toxin type A, botulinum toxin type B, botulinum toxin type C, botulinum toxin type D, botulinum toxin type E, botulinum toxin type F, and botulinum toxin type G.

11. A stabilized botulinum toxin composition, wherein said stabilized botulinum toxin comprises
a botulinum toxin complex or a reduced botulinum toxin complex; and
a polypeptide having a sequence according to SEQ ID NO. 2.

12. The stabilized botulinum toxin composition according to claim 8, wherein said polypeptide is non-cdvalently associated with said botulinum toxin complex or reduced botulinum toxin complex.

13. The stabilized botulinum toxin composition according to claim 8, wherein said polypeptide is covalently attached to said botulinum toxin complex or reduced botulinum toxin complex.
